(19) United States
(12) Patent Application Publication
(10) Pub. No.: US 2010/0137159 A1
(43) Pub. Date: Jun. 3, 2010

(54) Simple Tests for Rapid Detection of Canine Parvovirus Antigen and Antibodies

(75) Inventors: Sanjay Kapil, Stillwater, OK (US); Shashidhara Marulappa, Stillwater, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(73) Assignee: The Board of Regents for Oklahoma State University, Stillwater, OK (US)

(21) Appl. No.: 12/606,451

(22) Filed: Oct. 27, 2009

Related U.S. Application Data
(60) Provisional application No. 61/108,918, filed on Oct. 28, 2008.
(60) Provisional application No. 61/145,793, filed on Jan. 20, 2009.

(51) Int. Cl. C12Q 1/70; C40B 30/04

(52) U.S. Cl. 506/9; 435/5

(57) ABSTRACT

Slide agglutination tests (SATs) and slide inhibition tests (SITs) provide rapid detection, quantitation and strain identification of red blood cell (RBC) agglutinating viruses such as canine parvovirus (CPV) in biological samples. The tests are rapid, low-cost, and easy to use. These tests do not require any expensive equipment and can thus be used to monitor infections and antibody titers under field conditions. The tests can be modified to detect results using fluorescence (FSAT). FSAT is useful for rapid high-throughput screening (HTS) of libraries of small molecules and/or chemical compounds to identify antiviral compounds useful for the treatment of diseases caused by emerging hemaglutinating viruses that infect animals and humans.

### CROSS REFERENCE TO RELATED APPLICATIONS

This application claims priority of U.S. Provisional Patent Application No. 61/108,918 filed Oct. 28, 2008; and U.S. Provisional Patent Application No. 61/145,793, filed Jan. 20, 2009, both of which are entitled “SIMPLE TESTS FOR RAPID DETECTION OF CANINE PARVOVIRUS ANTIGEN AND ANTIBODIES,” the contents of which are hereby incorporated by reference.

### BACKGROUND OF THE INVENTION

1. Field of the Invention

The invention generally relates to rapid, low-cost robust tests for detecting red blood cell (RBC) agglutinating viruses. In particular, the invention provides a slide agglutination test (SAT) and a slide inhibition test (SIT) which provide rapid detection, quantitation and strain identification of RBC agglutinating viruses such as canine parvovirus (CPV).

2. Background of the Invention

Canine parvovirus (CPV) is the number one viral cause of puppy enteritis and mortality (8). Unique properties of CPV make it an emerging and reemerging pathogen of dogs worldwide (2, 5, 16, 17). Parvoviruses have a single-stranded DNA genome of 5000 bases with a hair pin structure (4). Parvoviruses have exceptional evolutionary ability (10). Parvoviruses are extremely stable in the environment and relatively resistant to disinfectants because these are non enveloped viruses (19). Canine parvovirus multiplies in the rapidly dividing cells in the crypts of the intestine leading to diarrhea and dehydration (4).

In a kennel environment, the availability of a large number of susceptible puppies, environmental stress and unique properties of CPV make an ideal scenario for rapid spread of CPV. Effective commercial modified live virus vaccines are available that vary in the genotypes (CPV-2, CPV-2b) of CPV in the vaccine. There is currently no commercial vaccine with CPV-2c in the vaccine. However, induction of active immunity in puppies is blocked by maternal immunity in the puppies (18). Stability of canine parvovirus in the kennel environment and excretion of large amounts of CPV by sick puppies can expose susceptible puppies to massive infectious doses of CPV. This CPV susceptibility window coincides with weaning in the age group of about 6-8 weeks of age. Eight weeks of age is the peak period in which large numbers of puppies succumb to CPV. Moreover, there are individual variations in the decay of the antibodies and induction of active immunity after vaccination directed by the genetic makeup (canine major histocompatibility antigens) of the puppies.

Several tests that have been used for rapid detection of CPV in fecal samples and CPV antibodies. These tests include tests based on immune-chromatography (15), latex agglutination (1, 20) and coagglutination (21). However, each of these tests has drawbacks such as a requirement for specialized equipment, high cost for the test, need for extensive training of personnel to use the test, length of time before results are obtained, etc. In addition, these tests require special reagents.

It would be clinically useful if there were rapid diagnostic tests available to 1) detect the amount and genotype of CPV in a biological sample; and 2) quantify and identify the type of antibodies against different CPV subtypes in a biological sample. This would be especially advantageous if such diagnostic test could be readily employed in the kennel environment at room temperature.

### SUMMARY OF THE INVENTION

The present invention is based on the surprising discovery that scaled down, microliter-size versions of hemagglutination and antibody inhibition tests, performed on a flat surface instead of wells, can be used to rapidly and accurately determine the presence of an RBC agglutinating virus, and the amount and type of antibodies against the virus in biological samples. The tests are robust and can be performed at a wide range of pH values. Accordingly, the present invention provides two rapid tests which, when used together, assess the presence and strain type of an RBC agglutinating virus of interest in biological samples. The tests provide instant results as soon as the reagents are mixed for detection of CPV antigen in feces and antibody quantification is serum. Without being bound by theory, the main principle of the tests is believed to be that, while they preserve the intrinsic hemaglutinating property of the virus, the flat design of the assay results in the reaction taking place in a very thin film, thereby totally eliminating the settling time for erythrocytes that is required in previously known assays (tube or plate agglutination tests). One test is a slide agglutination test (SAT). SAT is used for the detection and identification of viral antigens. The other test is a slide inhibition test (SIT). SIT is used for viral antibody typing. Both of the tests are instantaneous, real time tests that are sensitive and quantitative for a viral type of interest. In addition, the tests are safe, economical, and easy to use, requiring only minimal equipment and training of personnel. In one embodiment of the invention, the viruses that are detected and characterized using the tests are canine parvoviruses. In this embodiment, the availability of the tests encourages timely use of vaccines in puppies based on correct determination of antibody decay in an individual puppy, and therefore helps to prevent CPV outbreaks. The tests also help to manage outbreaks of CPV in kennels when they do occur, with minimum training of kennel personnel.

### BRIEF DESCRIPTION OF THE DRAWINGS

FIGS. 1A and B. Schematic representation of a slide test of the invention. A, shows a possible arrangement of rows of circles within which reaction components are mixed on a flat substrate; B, shows results of a hypothetical assay with positive (circles 1B, 1C, 2A and 2C) and negative (circles 1A, 1D, 2B and 2D) results.

FIGS. 2A and B. Schematic representation of results from Slide Agglutination Test (SAT): A, Canine parvovirus positive sample showing agglutination (clumping) of porcine erythrocytes by slide agglutination test (SAT positive); B. Canine parvovirus negative sample showing lack of agglutination of porcine erythrocytes by slide agglutination test (SAT negative).

FIGS. 3A and B. Schematic of test that uses surface fluorescence for detection and confirmation of virus binding on the surface of erythrocytes.

### DETAILED DESCRIPTION

The invention provides small footprint, rapid, and easy to use assays for 1) detection of an RBC binding virus; and 2) typing of antibodies to a virus. The tests are light weight and easily disposable after use, an advantage since these days biological waste disposal is expensive for glass or plastic tubes or plastic plates. The tests do not require the use of expensive, specialized instruments and users need not undergo highly advanced technical training in order to use and interpret the test results. The tests provide an overall picture of the extent of exposure and/or infection and/or immune response of an animal or individual with respect to an RBC-binding virus of interest. The tests are well-suited for field deployment in remote, under developed areas for emerging viruses of animals and/or humans, as they do not necessarily require much cold or refrigeration. Kits for carrying out the tests can be assembled with minimal use of expensive reagents.

One test is a slide agglutination test (SAT). SAT is used to detect the presence of viral antigens in a sample. The test is based on the fact that many viruses have surface or envelope proteins that enable them to attach to molecules (usually transferrin) present on the surface of red blood cells. The RBCs in a solution in which such viruses are present (at a sufficient concentration) bind together (agglutinate) and form a lattice with the viruses. Samples with agglutinated RBCs can be distinguished from those in which RBCs are not agglutinated by visual inspection. In addition (and optionally), by serially diluting a virus suspension and adding a standard amount of RBCs, an estimation of the number of virus particles in a sample can be made, since a minimum virus concentration is necessary to initiate agglutination. While these principles were previously known, prior art versions of such tests typically required at least a 120-240 minute incubation (i.e. 2-4 hours) after combining the viral samples with RBCs before the results could be read. What was not previously appreciated was that microliter amounts of virus sample and RBC solution could be combined in a cold, flat plate version, and that accurate, reliable results could be obtained by reading the results (e.g. by visual inspection) after only about one minute or less. High concentration samples are positive as soon as the reagents are brought together, even at room temperature. SAT also performs well at room temperature and at 37° C.; however, the test is easier to read when cold slides (e.g. 4-8° C.) are used. The standard hemagglutination test requires plastic plates with cups and/or wells that are “U” or “V” shaped. The plates are typically used at room temperature and then cooled in the refrigerator during incubation. In contrast, according to one embodiment of the present invention, a pre-cooled glass plate is used. That, coupled with eliminating the need for agglutinated erythrocyte settling, provides results that are, for all practical purposes, instantaneous. The use of microliter amounts of samples makes possible a small footprint assay that is inexpensive to manufacture, easy to store and dispose of, and which allows many samples to be tested quickly in a relatively small facility. In fact, the tests can be carried out on cards made of paper, thus simplifying disposal since the cards can be burnt after use with canine parvovirus samples.

The other test is a slide inhibition test (SIT). The SIT test is based on the ability of viral antibodies to bind to a virus and to prevent its ability to bind to RBC receptors, thereby blocking agglutination of RBCs. SIT is used to detect the presence and type (and optionally, the amount) of viral antibodies in a sample. To carry out this test, a virus of interest is exposed to a putative source of antibodies to the virus (e.g. serum, colostrum, etc.) under conditions conducive to and for a time sufficient to allow binding of the antibodies (if present) to the virus. Then, RBCs are exposed to the virus. If antibodies to the virus were in the putative source, then the virus in the mixture will have been blocked by the antibodies and will be unable to induce RBC agglutination. Conversely, if no antibodies were present, then RBC agglutination will be induced by the virus. Again, the difference between agglutination and lack thereof can be readily detected by visual inspection. Further, by testing several different viruses or virus types, more specific information can be obtained. For example, CPV antigenic variants or genotypes CPV 2a, CPV 2b, CPV-2c, etc., can each be tested and distinguished from one another, and the presence of one (or more) variants in a sample can be detected. In addition, by using a standard amount of virus, a standard amount of blood cells and serially diluting the antibody source, it is possible to identify the minimum inhibitory concentration (the highest dilution which inhibits hemagglutination) and thus quantitate the amount of antibody in the sample. While the general principles of antibody inhibition of viral-induced RBC agglutination were previously known, it was not previously appreciated that reliable results could be obtained by carrying out the reactions on a microliter scale and for a very short period of time (e.g. about one minute or less) in a cool, flat plate version.

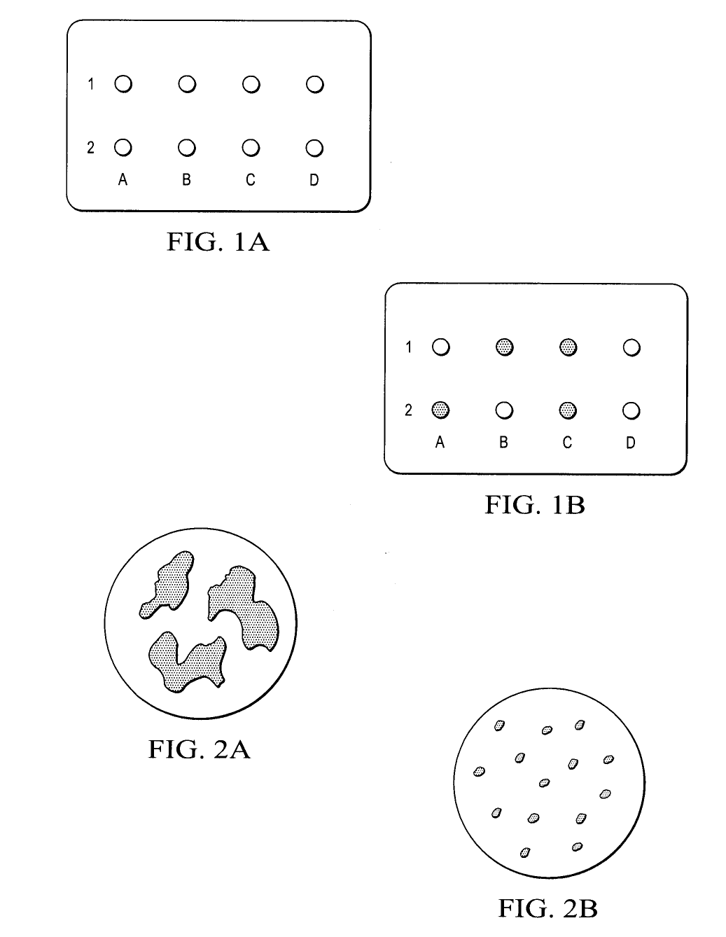

By “microliter amounts” or on a “microliter scale”, we mean that the total quantity of liquid samples used to conduct the slide tests, after mixing (i.e. the reaction mixture), is less than about 1000 μl, preferably less than about 500 μl, more preferably less than about 250 μl, even more preferably less than about 100 μl, and most preferably about 75 μl or less. In some embodiments, the total quantity of liquid will be about 70, 65, 60, 55, 50, 45, 40, 35, 30, 25, 20, 15, 10, or 5 μl with a typical amount being about 50 μl. In some embodiments of the SAT assay, in order to arrive at a total reaction mixture volume of e.g. 50 μl, 20 μl of a sample that may contain virus is combined with 20 μl of an RBC solution and 10 μl of a suitable buffer. Alternatively, 25 μl of a sample that may contain virus may be combined with 25 μl of an RBC solution, with no added buffer; or 20 μl a sample may be combined with 30 μl of an RBC solution, etc. For a typical SIT assay, which involves two steps (incubation of antibody source with virus, and subsequent incubation with RBCs) similar quantities may be used to arrive at a final volume of less than about 50 μl or less of antibody source plus virus solution plus RBC solution. The final volume is in the microliter range, and the amounts are standardized within a given testing regime to achieve results that can be read, e.g. by visual observation, within a short time frame, preferably less than 1 minute. For ease of use by personnel, standardization of the test may be done so that identical quantities of sample and RBC solution are added to a reaction mixture. For example, kits comprising substrates with premarked or preformed circles or impressions/indentations (or even locations surrounded by a ridge to prevent spillover of the liquid contents) may be provided. Such substrates may be referred to as devices. Such designated locations or indentations would be designed, numbered or otherwise labeled and premeasured to accommodate microliter quantities of liquids as described herein, to allow adequate mixing of a reaction mixture, without mixing between individual reaction mixtures. For example, if circular reaction spaces are drawn on an essentially flat, planar substrate such as a glass slide, the internal diameter of such circles will typically be about 5 cm or less, preferably about 4 cm or less, more preferably about 3 cm or less, and most preferably about 2 centimeters or less, and possibly as low as about 1 cm or less (e.g. about 1.9, 1.8, 1.7, 1.6, or 1.5 cm or less). For example, in one embodiment, such circles have an inner diameter of 1.9 cm, and can readily accommodate a liquid volume of about 50 μl. Such a marked slide is depicted schematically in FIG. 1A, where a slide premarked with a total of 8 circles (in two rows of four circles each) is depicted.

By a “short time frame” or “instant” tests, we mean that the incubation time of reaction components after being mixed together (i.e. the time after mixing at which the results can be observed) is about 5 minutes or less, preferably about 4 minutes or less, more preferably about 3 minutes or less, even more preferably about 2 minutes or less, and most preferably is about 1 minute or less. For example, the time may be about 75 seconds, 70 seconds, 65 seconds, 60 seconds, 55 seconds, 50 seconds, 45 seconds, 40 seconds, 35 seconds, or even about 30 seconds or less. In the previously known plate or tube versions of the HA test, elution of erythrocytes has been observed, making the tests unstable and difficult to read. This has not been observed in the flat substrate tests described herein, the results of which are thus more easily read.

All components of the reactions mixtures of the invention may be used either directly if possible, or more usually, with some prior preparation. For example, samples may be suspended or diluted in buffers, e.g. in buffers that enhance (or at least do not inhibit) the reactions on which the tests are based. The exact conditions will depend on the type(s) of virus(es) that is/are being detected, as some viruses bind RBCs only or optimally at certain pH values, and others at certain ionic strengths, etc. Hence, the pH, inoic strength, composition, etc. of the suspension buffers can be adjusted for each virus to take these factors into account, by means that are known to those of skill in the art. Conversely, by subjecting an unknown sample to varying test conditions, it may be possible to further characterize the virus in a sample. For example, if a carnivore fecal sample is tested with porcine erythrocytes and agglutinates preferably at pH 6.5, then it is a feline pan-leucopoenia virus (feline parvovirus). If the virus agglutinates at a wider rage of pH values, then it is canine parvovirus. Further, the pH and temperature are also a measure of the nature of the viruses involved. If a virus is influenza A and agglutinates turkey or chicken erythrocytes, then very likely the virus is reacting with seasonal human influenza A viruses (H1 and H3). If avian influenza A viruses are involved, they preferentially agglutinate erythrocytes from horses and cows. Thus, this simple test can help narrow the type of viruses involved and their interaction with respective receptors.

With respect to the pH values at which the tests of the invention are performed, in some embodiments, for detection of canine parvovirus the pH should be at least about 7.2 whereas for detection of feline panleukopenia virus or feline parvovirus the pH should be about 6.5. Alternatively, as described in Example 5 below, the tests, particularly the SAT test, may advantageously be carried out at multiple pH values in order to characterize the virus further, e.g. at pH values in the range of from about 5 to about 9, and preferably at pH values of 6.5, 7.2 and/or 8.0. Without being bound by theory, viruses that can cause agglutination of RBCs at a wide range of pH values (e.g. as low as 3 and greater than 7.5) may be more virulent than viruses that cannot. Agglutinating activity at this wide range of pH values likely indicates great flexibility in virus-receptor interaction, and thus increased ability of the virus to infect cells in a wide range of environments (e.g. both the small and large intestines). When a feline parvovirus isolate (08120386) was compared to a CPV-2c isolate (08110650), it was found that the CPV-2c isolate agglutinates porcine RBCs at pH values ranging from about 3 to about 8. In contrast, the FPV isolate caused agglutination well only at pH 6.5, and less well at pH 4.0 and 5.0 (data not shown). Thus, use of the tests described herein at a broad range of pH values can help to identify and characterize well-adapted CPV strains capable of infecting cells along the entire length of the intestines. Such strains are likely to be highly virulent and thus there identification can be of importance when evaluating potential vaccine components, the best response to a viral outbreak or epidemic, etc. In addition, these findings have implications for sensitive detection and correct measurement of antibody titers against a virus using the SIT test, e.g. against CPV-2. It was not previously appreciated that CPV-2 isolates differ in their pH tolerance, requirements or optima for RBC agglutination. Carrying out SAT and/or SIT tests on a sample at a wide range of pH values has the potential to provide valuable information (e.g. concerning virulence) that would otherwise be missed if conventional, single pH value tests are used. When the tests of the invention are carried out at low pH values, (e.g. pH 3-4) it should be realized that at these pH values, porcine erythrocytes lyse in about 20 minutes. Thus, for such assays, small batches of 2.5% erythrocytes should be made to check virus binding to RBCs in this low (acidic) pH range. For general diagnostic purposes, testing in the pH range of from about 5 to about 8 is usually sufficient, and within this pH range, porcine RBCs are usually stable. Generally, all viral isolates should be tested at least at pH values of 6.5, 7.2 and 8.0, in which case phosphate buffered saline (e.g. Sorrensons's PBS) buffer is preferred, since PBS is known to have good buffering capacity in this pH range (e.g. from about pH 5 to about pH 8). However, testing at lower pH values (e.g. pH 5.0 or thereabouts) can also be carried out if desired by using a suitable buffer. For example, citric acid/sodium citrate buffer provides good buffering capacity in the pH range of about 3.0 to about 6.0 and works well for the tests of the invention. As can be seen, the assays described herein are very robust in that they can be easily and successfully carried out at a wide range of pH values, and thus used to obtain valuable information for the characterization of a virus.

The RBCs that are employed in the assay may be from any suitable and convenient source, examples of which include but are not limited to RBCs from animals (e.g. pigs, dogs, cats, etc.) or from humans. The source of erythrocytes depends on the virus to be detected. Typically, porcine RBCs are employed for CPV-2 because they provide high sensitivity. However, if porcine erythrocytes are not readily available, then canine and/or feline erythrocytes can be used. Porcine erythrocytes however will provide similar or slightly higher sensitivity, and were used in the examples presented herein. The fresh RBC samples can be stored refrigerated (e.g. at about 0 to 10° C., or preferably from about 2 to about 8° C.) for up to about 72 days and still perform well in the tests. For use, the RBC sample should be adjusted to a concentration of about 2.5%. Samples which are stored for extended periods of time (e.g. refrigerated for more than one week) may exhibit hemolysis. If this is the case, the RBCs can be washed to remove hemolysis products and then diluted appropriately. If necessary, RBCs may be stored at room temperature (e.g. from about 20 to about 25° C.). In this case, low levels of sodium azide (e.g. from about 0.05% w/v to about 0.00005%, w/v) may be added to the RBCs to aid in preservation. The concentration selected depends on adjusting conditions prevent spoilage of erythrocytes and endocytosis of transferring receptors, but not to chemically alter the transferrin receptors on the surface of the erythrocytes. Moreover, the sodium azide is a very low molecular mass molecule and can be easily dialyzed out against e.g. PBS (pH 7.2, storage buffer) prior to use of the erythrocytes, further reducing the concentration of sodium azide in where this might interfere with the test, if this is suspected. The erythrocytes are selected to achieve the maximum sensitivity. In one embodiment, 2.5% porcine erythrocytes are used. Further, the RBC sample will typically include serum such as fetal bovine serum (FBS), in a concentration of less than about 10% and preferably less than about 5%. In one embodiment, the FBS concentration is about 2%. Also, the RBCs may optionally be treated with antibiotics and/or antifungal agents to aid in their preservation, whether stored refrigerated or at room temperature.

Various buffers may be used when conducting the tests, for example, to perform serial dilutions of a sample, to bring the total volume of a sample to a desired final volume, etc. Suitable buffers generally include those that are known to those of skill in the art, including phosphate buffered saline (PBS). In addition, the buffers may be adjusted (e.g. pH, ionic strength, etc.) to optimize the rate of interaction of the assay components, in particular for the SIT assays as described below. For example, when analyzing fecal samples from dogs, a 0.2 M PBS solution, pH 7.2 may be used. However, if the fecal sample is from a cat, then a solution with pH 6.5 is preferred. In addition, one requirement of the tests is that the NaCl concentration should be physiologically compatible, e.g. about 0.9%, in order to prevent lysis of the erythrocytes.

Generally, carrying out the small volume tests involves providing a suitable surface or support on which the requisite assay components can be placed, mixed, allowed to incubate, and observed to note the results. These components include: a suitable quantity of biological (i.e. unknown or experimental) sample; a suitable quantity of RBC-containing sample; and for the SIT assay, a suitable quantity of known viral sample (antigenic type positive controls). Optionally, a suitable quantity of buffer may be included e.g. to bring the reaction mixture to a desired final volume, to adjust the pH and/or iconic strength in order to facilitate binding reactions, etc. In some embodiments of the invention, the SIT and SAT tests are carried out on separate supports, while in other embodiments, both test are carried out on a single support. The supports that are used are generally substantially planar to aid in retention of the reaction mixtures within a specific bounded region. In one embodiment, the support is a glass microscope slide, although this need not always be the case. In some embodiments, a card (e.g. a disposable card) that is impermeable to liquid (e.g. made of or coated with various polymers, plastics or metals) may be used. Any substantially non-absorbent planar surface of any convenient shape may be utilized. The surface may be treated e.g. with a hydrophobic substance, in order to promote retention of droplets in a circumscribed area. Typically, the planar surface of a support or substrate will be marked at spaced apart intervals to indicate where reaction mixtures are to be located, i.e. the reaction positions. Generally, such locations will be indicated as e.g. a circle, rectangle, dot, or other shape that is drawn, printed or etched onto the support surface (or within or on the underside of the support material, but visible on the surface on which the droplets are deposited). The locations may also be indicated by an indentation in the surface. The support may also include other markings such as numbers and/or letters e.g. to differentiate spots or rows from one another and to allow the user to keep track of the samples and to locate a reaction mixture of interest. Such devices may be included, for example, in a kit. In one embodiment, oval-shaped spots were used. Various sizes of such shapes may be used. For example, if a viral system has typically a lower titer in the specimen, then a larger area accommodating larger amounts of reagents can be used. Or, if the infection produces very high titers, as is typically the case for CPV-2 infections, then smaller volume circles, ovals, etc. can be employed. The color of the surface may be used to provide better contrast and higher visibility of the section, for example, a shiny white surface.

To carry out a test, the user obtains a suitable support, which generally will have been stored frozen or ice cold e.g. at 4° C., and will generally will be kept cold e.g. on ice, throughout the assay. The reagents and samples will also be cold, e.g. stored on ice, prior to and during use. This is in part because enzymes that degrade RBCs (e.g. by removing transferrin from the RBC surface) may be present in biological samples and otherwise could interfere with the assay results. The erythrocytes are kept cold to prevent lysis, and/or a preservative such as sodium azide (0.1%) may be added for preservation. Sodium azide also prevents endocytosis of surface transferrin molecules making erythrocytes more stable and checking environmental contaminants. The primary samples (e.g. a fecal sample, blood sample, a swab from which material has been eluted, etc) may be used directly and/or diluted prior to use. For the SAT test, suitable microliter quantities (microdroplets) of sample, RBC solution and, optionally, additional buffer, are allocated onto the surface of the support within or close to one of the indicated positions, e.g. within a circle, rectangle, oval, etc. marked on the support. In preferred embodiments, the shape is a circle or oval. If dilutions have been done, multiple individual aliquots (one per dilution) will be placed within multiple circles/ovals. Further, each reaction is generally done in duplicate or triplicate, with suitable positive and negative controls. However, for a field test, one dilution may be sufficient. The microdroplets for a single reaction are all deposited within the boundaries of one position on the plate, and initially are adjacent to but not in direct contact with one another. The precise order of addition of the reaction mixture components to the plate is generally not crucial. Once all components are present, the reaction components within each designated area (e.g. within a circle) are rapidly mixed for about 60 seconds or less, preferably for about 45 seconds or less, more preferably for about 40 seconds or less, e.g. for about 35, 30, 25, 20, 15, or 10 seconds. In one embodiment, mixing is carried out for 30 seconds. However, some strong samples will react within only a few (e.g. about 1-5 or 5-10) seconds. Mixing of the reaction mixture components may be carried out by any suitable means, e.g. by simply using a toothpick (e.g. a disposable toothpick), the tip of a pipette, etc. For e.g. field use, the user can simply mix by manually rotating the slide to allow mixing for 30 seconds or 1 minute and read the reaction result. The final volume of reactants at a reaction position and the distances between reaction positions on the support should be such that reaction components can be mixed without mixing with or flowing over into reaction components or mixtures in adjacent reaction positions. The slide is then allowed to incubate on ice (or refrigerated) for the requisite period of time, e.g. for about one minute or less. Afterwards, the user visually inspects the slide and notes the results (agglutination or no agglutination). This is illustrated schematically in FIG. 1B, which shows a hypothetical result in which spots 1B, 1C, 2A and 2C (filled in circles) are positive for agglutination, and spots 1A, 1D, 2B and 2D are negative for agglutination (empty circles). If results are equivocal, in that partial clumping of RBCs may be observed, the samples may be retested (with less dilute sample, if possible, or with higher concentrations of reactants such as RBCs for SIT and SAT, and viruses for SIT); or incubated longer (e.g. from about 1-15 minutes), to achieve unequivocal results. It is noted that this reaction also proceeds at room temperature and can be carried out at 37° C., but less efficiently. Generally, the reaction is carried out at a temperature of about 1° C. to about 25° C., preferably from about 2° C. to about 20° C., more preferably from about 3° C. to about 15° C., even more preferably from about 4° C. to about 10° C., and most preferably at about 5, 6, 7, 8 of 9° C. Generally, 5° C. is preferred, or refrigeration if available.

For the SIT assay, the procedure is similar except that the unknown sample (which may or may not contain antibodies to a virus of interest) and the virus of interest (which may be referred to as a known viral standard) must be mixed first, allowed to react for about 1, 2, 3, 4, or 5 minutes (reaction for about 1 minute is preferred, although higher sensitivity may be achieved with longer incubation times), and then mixed with RBC detection solution and allowed to incubate further for about 1 minute or less, as described for the SAT assay. It is noted that multiple differing standard viral samples (e.g. different viruses, different viral strains or subtypes, etc.) may be loaded onto discrete locations on a plate and each may be tested using individual aliquots of a single biological sample (e.g. a blood sample). In an alternative embodiment, for the SIT reaction, viral samples may be preloaded onto the substrate and dried or lyophilized in order to fix the virus standards to the plate (disposable card, slide, etc.). Cards with lyophilized antibodies can be sealed in, e.g. ziplock bags and have a very long shelf life at refrigerator temperatures if a desiccant is placed in the bag. Since the result of a SIT test is also either agglutination or no agglutination, reading of the results is the same as that described above for the SAT test.

Generally, the test results are noted by visual observation of the reaction mixture droplets on the slide. No equipment is required. However, if more detailed information is desired, the results may also be viewed, for example, with a microscope or magnifying glass at 4×, 10× or 100× magnification. In one embodiment, a hand held 5× magnification device was found to be suitable. In addition, while reliable results are obtained after only about one minute of incubation, the plates may be incubated longer (e.g. up to about 5 minutes) if desired.

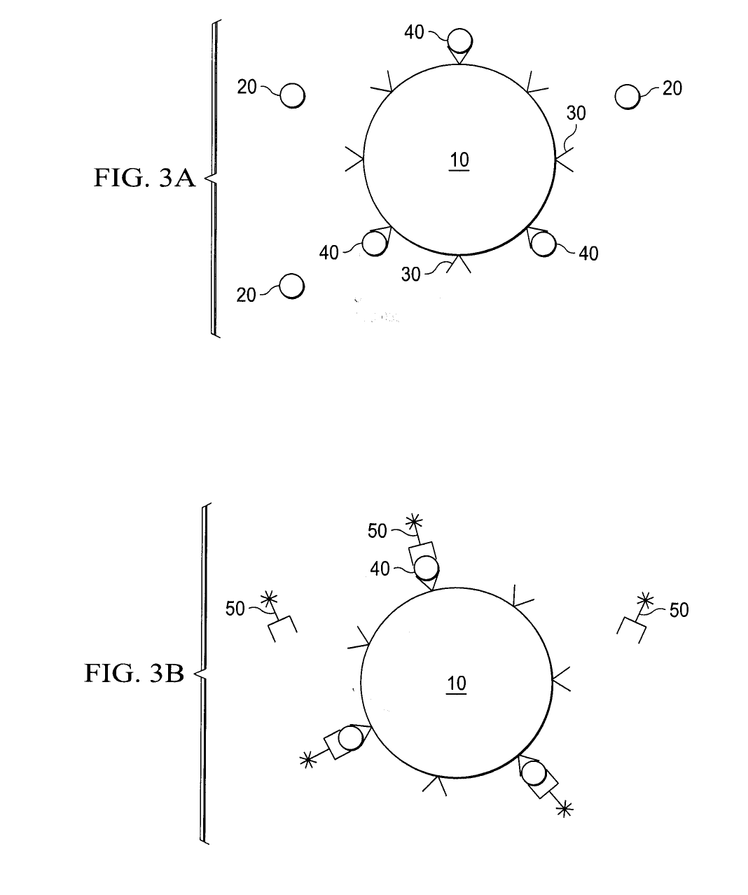

In yet other embodiments of the invention, the viruses of interest, especially CPV viruses, are detected using cell surface immunofluorescence (“surface immunofluorescence” or “surface fluorescence” (SF) using the SAT methods of the invention. In surface fluorescence, the cell surface molecules of living cells used in the technique are biologically functional and retain their biological activity. In the ease of RBCs, transferrin on the surface is exposed and not denatured, and will thus interact with e.g. parvovirus. This embodiment is illustrated schematically in FIGS. 3A and B. With reference to FIG. 3A, RBCs 10 are exposed to a sample which purportedly contains a virus of interest 20. If viruses of interest 20 are present in the sample, they bind to transferrin receptors 30 on the surface of erythrocytes 10, causing the RBCs to agglutinate and precipitate, so that they stick to the substrate on which the test is being carried out. Bound viruses 40 are shown in FIG. 3A. Subsequently, with reference to FIG. 3B, RBCs 10 are exposed to fluorescently labeled antibodies 50 that are specific for virus of interest 20, e.g. a particular strain of CPV or other virus that causes agglutination of RBCs. Known fluorescent labels such as fluorescein isothiocyanate (FITC) may be used to label the antibodies by methods that are known in the art. In one embodiment, direct fluorescent anti-CPV-2 antibody FITC conjugate is employed (such as that which is commercially available from VMRD Inc., (Pullman, Wash.). If virus of interest 20 is present in the sample, viruses bind to the RBCs and fluorescent antibodies 50 would then attach to bound viruses 40, thereby (indirectly) fluorescently labeling RBC 10. Typically, excess sample and unbound fluorescent antibodies are removed from the reaction (e.g. by washing) and the RBCs that remain at the site of reaction are then examined in order to detect associated fluorescence. Methods of detecting fluorescence are known and generally include exposing a sample to (i.e. interrogating a sample with) a light source of suitable wavelength (e.g. ultraviolet light), and detecting and/or measuring (quantifying) the fluorescence that is emitted using a suitable fluorescence detector. If an RBC sample displays fluorescence, this indicates that the sample to which it was exposed contained a virus that 1) bound to the RBC surface and 2) was recognized and bound by the fluorescently-labeled specific antibody. Thus, the presence of fluorescence is a positive result (the virus of interest was in the sample), and the absence of fluorescence is a negative result (the virus of interest was not in the sample). Those of skill in the art will recognize that the degree of fluorescence that is detected in a positive sample correlates with the amount of virus in the sample, with higher levels of fluorescence indicating higher titers of virus, and lower levels of fluorescence indicating lower titers of virus. SF works well to detect viruses such as CPV in feces with high sensitivity and high specificity.

One problem that may be encountered when using fluorescent labels in this manner is endocytosis, i.e. the RBC may engulf (internalize) the virus particle after it binds to the RBC transferrin receptor so that the fluorescently labeled antibody cannot bind to the virus, possibly resulting in a false negative result. Generally, the longer the incubation time of the virus with the RBC, the greater the chance of endocytosis. This problem can be addressed, for example, by shortening incubation times and/or by altering reaction conditions to be less favorable to or to inhibit endocytosis, e.g. by adding inhibitors such as sodium azide (NaN₃), as described in detail in Example 3.

It is not possible to use fluorescent antibody testing on feces or fluids since viruses cannot be seen with a fluorescence microscope. However, for tissues or cell samples, surface fluorescence detection provides distinct advantages: 1) SF provides additional specificity and enhanced sensitivity to verify SAT tests; 2) if there is a rare virus (e.g. a CPV-2 virus) that does not cause agglutination, SF will still provide a true indication of the presence of the virus in the sample. (Agglutination is a two-step process: virus binds to the erythrocytes and then cross-links the erythrocytes. Some weak viruses can bind but not crosslink. Thus, a weak sample or a non-agglutinating CPV will bind on the erythrocyte surface and not be washed off, even though clumping/cross-linking did not occur, or occurred only to a limited extent). 4) SF can be done using ultra-low total volumes for detection, e.g. about 50 μl or less; about 40 μl or less; about 30 μl or less; about 20 μl or less; about 10 μl or less, or even about 5 μl or less; and 5) SF can be readily applied in high-throughput screening (HTS) techniques. With respect to the latter advantage, SF can be used to identify antiviral compounds, for example, anti-CPV-2 compounds. Generally, known virus samples are exposed to candidate antiviral compounds prior to exposure to a virus sample in order to ascertain whether or not the compound can inhibit binding of the virus to transferrin. If the ability of a virus to bind transferrin is blocked by the compound, the SF result will be negative. If the compound does not affect the viruses ability to bind transferrin, the SF result will be positive. This facet of the invention is explained in more detail in Example 4.

The invention provides two tests, SAT and SIT, and, in general, the two tests will be used together, and may also be used in conjunction with SF. In other words, both tests will generally be performed side-by-side, or consecutively, especially when an individual or individuals is/are being tested for the presence of a viral infection. However, this need not always be the case, and the SIT and SAT tests may be used individually if desired. The type of information provided by each test differs and various strategies for the use of the tests may be developed. For example, if a dog in a kennel develops diarrhea, a fecal sample may be tested using SAT to determine whether or not the presence of CPV is likely (CPV is the major cause of diarrhea in a kennel environment). Kennel personnel may or may not wish to define more closely the type of CPV by carrying out a SIT test for that individual. However, it would be likely that other dogs (especially puppies) in the kennel should at least be tested using SIT to determine whether they have sufficient antibodies to ward off a potential infection, and if not, vaccination could be undertaken. The assessment of fecal samples might or might not be of value for dogs that did not yet display any symptoms of disease such as diarrhea. However, under most circumstances, it is likely that both tests would be carried out, since they are very inexpensive and easy to use, and more information is provided.

The viruses that are detected by the tests and methods of the invention are viruses that are capable of binding to RBCs, examples of which include but are not limited to parvovirus (e.g. various types of mammalian parvoviruses such as canine parvovirus, feline panleukopenia virus, mink parvovirus, etc.); coronaviruses such as severe acute respiratory syndrome (SARS) virus; human immunodeficiency virus (HIV); influenza viruses, Newcastle's disease virus, infectious bronchitis virus, adenoviruses such as adenoIII virus, etc. In one embodiment, the virus is a canine parvovirus.

The tests of the invention are carried out using suitable samples that are generally obtained from a known individual that is being tested. In some embodiments, the samples are obtained directly or nearly directly from the individual. For example, for the SAT test, which detects the presence of the virus, suitable “direct” biological samples include but are not limited to fecal samples, tissue samples (e.g. tongue swabs, buccal swabs, and small intestine samples), rectal swabs, etc. For the SIT test, which detects the presence/absence of antibodies to one or more viruses of interest, suitable biological samples include but are not limited to blood and/or serum, saliva, plasma, bitch colostrum, etc. Of note, about 97% of antibodies are transferred from the bitch to her puppies by colostrum; thus colostrum antibodies titers can be measured. Any biological sample that may contain or is suspected of containing a virus of interest (for SAT) or antibodies to a virus of interest (for SIT) may be assayed, subject to proper preparation, as will be understood by those of skill in the art. For example, because the tests of the invention are liquid based tests, some samples may first be suspended in a liquid medium (e.g. a suitable buffer that does not cause erythrocyte lysis), or viruses may be eluted from the sample into such a medium, etc. Further, the samples may be obtained from either living or deceased individuals.

Those of skill in the art will recognize that the tests of the invention are also useful for more generalized tests that are not for individual subjects, e.g. for testing swabs or swipes of areas or articles that might be contaminated with virus (e.g. work surfaces, articles of clothing, dishes and utensils, etc.). In such cases, the result of the tests can be used to determine whether or not an area is sufficiently sterile to allow individuals susceptible to viral infection to be in the area (e.g. a kennel, veterinary clinic, room in a hospital or nursing home, food preparation areas, operating theater, seats in an airplane, animal transportation cages and carriers, examination tables, etc.). If evidence of viral contamination is found, the area can be thoroughly cleaned. In addition, the tests may be used to evaluate various disinfectants for their antiviral efficacy.

In another embodiment, the invention provides a rapid method or test for assessing the virulence of a virus that is capable of agglutinating RBCs. The ability of a viral isolate to agglutinate RBCs at higher temperatures is a direct and easy measure of its ability to bind to transferrin receptor. Thus, in this embodiment of the invention, the ability of an isolate to agglutinate RBCs is tested, using the SAT methods described herein, at various temperatures, e.g. about 37, 38, 39, 40, 41, and 42° C. The temperatures at which these types of test are carried out generally should not exceed about 42° C. or erythrocytes may lyse. The core body temperature cannot exceed about 43° C. so there is no need to test above 43° C. By observing the ability of isolates to agglutinate RBCs within a range of increasing temperatures, the potency or virulence of the virus can be assessed. The assessment is based on the positive correlation between the ability of a virus to bind transferrin and its virulence. Viruses that bind to transferrin with a high avidity generally tend to retain the ability to do so (and thus to agglutinate RBCs) even at elevated temperatures, and are thus likely to be more virulent than viruses that bind only at lower temperatures. For example, if three isolates are compared, the isolate that is able to agglutinate RBCs at temperatures up to and including 40° C. is likely more virulent than the isolate that can agglutinate RBCs at a maximum temperature of 38° C., and the virus that causes agglutination at temperatures up to 42° C. is likely the most virulent of all three isolates. Those of skill in the art will recognize that the precise temperatures at which the tests are carried out will vary from situation to situation, and will depend upon and can be tailored to the viruses being tested by altering the conditions under which the test is carried out, e.g. by adjusting the pH, buffer type, etc. so as to provide a suitable convenient test. While the SAT test in particular performs well at room temperature, or at temperatures up to about 37° C., the test is easier to read when carried out at cooler temperatures (e.g. 4-6° C.) because the clumps of erythrocytes are larger. In addition, the tests of the invention may be standardized and scales or degrees of virulence may be established. In some cases, this standardization may be correlated with other known measures of virus-transferrin binding, so that, for example, a value or range of values determined by the tests of the invention is established to be equivalent to a value or range of values (e.g. Ki, Km, etc) from other virus-transferrin binding assays. All such variations are encompassed by the present invention.

In yet another embodiment, the virulence of a viral isolate may be assessed by determining the speed with which it causes RBC agglutination. Measuring the speed at which agglutination occurs (under standardized conditions) is an indirect measure of the ability of the virus to bind to transferrin. Thus, when comparing viral isolates, agglutination times can be compared, and viruses with faster agglutination times are likely to be the most virulent. For example, if three isolates are compared and the agglutination times determined, (typically under conditions that result in agglutination times in the order of about 1, 2, 3, 4, or 5 minutes, or even smaller time intervals of about 10, 15, 20, 25, 30, 35, 40, 45, 50 or 55 seconds), then an isolate that requires 2 minutes to cause agglutination is likely less virulent than an isolate that causes agglutination in only 1 minute, and an isolate that requires only 15 seconds is likely markedly more virulent than either of the other two. The amount of virus will be kept the same i.e. constant. Those of skill in the art will recognize that the precise time intervals that are required will vary from situation to situation, and will depend upon and can be tailored to the viruses being tested, by altering the conditions under which the test is carried out, e.g. by adjusting the temperature, pH, buffer type, etc. so as to provide a suitable convenient test. In addition, the tests may be standardized and scales or degrees of virulence may be established. In some cases, this standardization may be correlated with other known measures of virus-transferrin binding, so that, for example, a value or range of values determined by the tests of the invention is established to be equivalent to a value or range of values (e.g. Ki, Km, etc) from other virus-transferrin binding assays. All such variations are encompassed by the present invention.

In yet another embodiment of the invention, the virulence of a virus can be assessed by combining the two virulence tests described above, i.e. by a combined assessment of the maximum temperature of binding and the speed of binding at several temperatures. These tests can be carried out separately and the results correlated, or, in some cases, the tests can be combined in a single reaction, i.e. the time of agglutination can be measured in each reaction that is carried out at each of several temperatures. The value of assessing virulence is, for example, so that informed choices about which viruses to closely monitor and/or include in a vaccine can be made. An outbreak of highly virulent virus should signal a need for prompt attention, whereas less virulent viruses may not require am immediate robust response. In addition, higher in vitro virulence isolates can be used to test or evaluate vaccine candidates for efficacy. Further, in vitro virulence can be further verified by dog virus challenge experiments in vivo.

The invention is further illustrated in the ensuing Examples, which should not be interpreted to limit the invention in any way.

### EXAMPLES

### Example 1

Canine parvovirus (CPV) is the number one viral cause of enteritis, morbidity and mortality in 8-week old puppies. Twin assays have been developed (a “slide agglutination test” or “SAT” for CPV antigen, and a “slide inhibition test” or “SIT” for CPV antibody) that are sensitive, specific, cost effective, and generic for all genotypes of CPV, and which provide instant results for CPV antigen detection in feces and antibody quantification in serum. These assays are useful for routine applications in kennels with large numbers of puppies at risk. The results of these assays are available in about 1 minute or less and do not require any special instrumentation. SAT/SIT technology will find applications in rapid screening of samples for other hemagglutination emerging viruses of animals and humans (influenza and SARS coronavirus).

### Materials and Methods

Clinical Samples: All the samples that were submitted to the Oklahoma Animal Disease Diagnostic Laboratory (OADDL) were from puppies that had a history of vomiting and diarrhea. These animals were suspect for canine parvovirus. Most animals had a history of hemorrhagic diarrhea and a few had yellow diarrhea with mucus. Fecal samples and intestinal tissues from CPV suspect dogs were prepared as 10% w/v suspensions in phosphate buffered saline (pH=7.2) for this study. A total of 23 clinical field submissions (intestinal contents/feces/intestinal tissue homogenates) were evaluated. In addition, cell culture supernatants (n=60) from dogs of known CPV status based on conventional tests were also tested. The CPV status of all the samples used in development of this assays was confirmed by conventional assays such as hemagglutination test and virus isolation followed by HA test for CPV quantification. The PCR (6) genotyping was done as described before (9). Conventional plate hemagglutination test (HA) was performed as described by Carmichael et al., 1980 (3). The samples were serially diluted two-fold in PBS (0.2 M) in V-bottom plates. First, 50 μl of PBS was added to each well of the plate. In the first column, 50 μl of sample (fecal suspension or cell culture supernatant) was added. The sample was mixed 5 times and 50 μl was transferred to the next well. Each sample was diluted from 1:2 through 1:4096. Then, 50 μl of PBS was added to each well. The hemagglutination test was performed using porcine erythrocytes (0.5%). The corners of the plate were tapped 4-5 times to mix the erythrocytes. The plates were covered with a lid and incubated at 4-7° C. for 2-4 hours. Positive agglutination was indicated by mat formation whereas button formation indicated a lack of agglutination. The titer was calculated as the reciprocal of the last well with agglutination. The results showed that samples showing agglutination of porcine erythrocytes are positive for CPV and other with no agglutination of porcine erythrocytes are negative for CPV-2.

Slide Agglutination Test (SAT): For SAT, the conditions of the test were standardized to obtain agglutination results within 30 to 60 seconds of mixing the reaction components. The buffer was the same as for the HA test, phosphate buffered saline (0.2 M PBS, pH=7.2). Cooled glass plates (American Scientific Products, catalogue Number M 6225) were kept in the freezer compartment of the refrigerator, cleaned and ready to use. The plates were enclosed in a large zip locking bag to prevent contamination of the wells. Each plate had 30 circles. For the assay, the plate was wiped with a paper towel to remove moisture and kept on a flat Styrofoam support to keep it cool during the procedure. 20 μl of unknown sample was added as a drop within a circle on the plate. 20 μl of porcine erythrocytes (2.5% v/v) suspended in 0.2 M PBS with 2% fetal bovine serum were also added as a separate drop in the circle. The total volume was made up to 50 μl by adding 10 μl of 0.2M PBS as a third drop inside the circle. The three drops were mixed with wooden tooth picks in a circular motion for 30 seconds. Canine parvovirus positive samples produced agglutination within one minute. Negative samples were homogeneous and showed no agglutination. However, all samples were further incubated in the refrigerator for an additional 5 minutes before the results were recorded and confirmed microscopically. Positive samples showed large clumps of agglutination (see schematic representation in FIG. 2A) and negative samples showed single erythrocytes homogeneously spread in the circle (see schematic representation in FIG. 2B). Partial agglutination was microscopically confirmed with smaller clumps of porcine erythrocytes. Weak fecal or cell culture samples can take up to 3 minutes to agglutinate in the refrigerator.

For determination of the amount of virus, CPV positive samples were diluted 2-fold in a V-bottom well plate (Linbro/Titertek, 96 U wells plate, ICN Biologicals, Inc., Aurora, Ohio). Using the SAT (Slide Agglutination Test) procedure, the results were recorded as agglutination (A), no agglutination (N) and partial-agglutination (P). The dilution of the sample that showed partial agglutination was recorded as 1 HAU (hemagglutinating unit). This convention and calculation were adopted from the hemagglutination-inhibition assays for CPV. The dilution that contained 1 HAU was divided by 8 to calculate the dilution containing 8 HAU of CPV (For example, fecal sample number 08071352 had a titer of 1:256 on SAT titration. A dilution of 1:32 will contain 8 HAU of the virus for SIT, as described below).

Slide Inhibition Test (SIT): For the SIT, a hyper immune serum (10 μl) from a CPV vaccinated dog (Galaxy vaccine, Schering Plough Animal Health. Elkhorn, Nebr.) was diluted 2-fold in a U-bottom plate with PBS (0.2 M, pH 7.2). Eight HAU units of CPV in 20 μl were added to the serum dilutions and the plate was incubated for 1 minute at 37° C. in an incubator. The serum was diluted up to 1:4096. Eight HAU of CPV isolates (CPV-2c, CPV-2b and a raccoon parvovirus) were used. A total of 5 CPV isolates were tested. CPV and serum dilution mixtures (30 μl) were added to the cool glass plate. 20 μl of porcine erythrocytes were added and the combined solution was immediately mixed with a tooth pick. The total reaction volume was 50 μl. The presence of CPV antibody in the serum was indicated by a lack of agglutination due to inhibition/blocking of the agglutination. The antibody titer was recorded as the inverse of the highest dilution that produced complete inhibition of erythrocyte agglutination.

Virus Isolation (VI): Canine parvovirus was isolated from clinical samples. Both intestinal tissues and fecal samples were used. Before inoculation, the samples were processed in two ways: one set was diluted to 10% v/v in PBS, centrifuged to remove the particulate material, filtered through 0.2 μm filter to remove the bacteria and about 1 ml was inoculated within one hour of plating the Crandall Reese Feline Kidney (CRFK) cell line. (The cells typically attach to flasks in about 30-45 minutes and they are then ready to inoculate; hence, they are ready to inoculate within about one hour or less.) The second set was extracted with an equal volume of chloroform and vortexed for 1 minute. The samples were centrifuged for 10 minutes and the clear supernatant inoculated on cells after filtration through 0.2 um filter. The cells were plated at the density of 40%. One hour after inoculation, minimum essential medium containing 5% fetal calf serum was added. The cells were observed daily for 6 days post inoculation for cytopathic effects, such as rounding, elongation of cells, and detachment. The cells were freeze-thawed and centrifuged and checked for CPV virus by hemagglutination tests using porcine erythrocytes (0.5%).

### Results

For standardization of SAT, three porcine erythrocyte concentrations were tried: 0.625%, 1.25%, and 2.5%. For all the assays, equal amounts of blood from 2 different pigs were collected in Alsever's solution and used within one week. The clarity and visibility of erythrocyte clumps based on their size was used as a criterion for selecting the erythrocyte concentration. Based on an experiment using a CPV positive and a CPV negative sample, 2.5% porcine erythrocytes gave the clearest positive and negative results were used throughout the tests. Three different final volumes of reaction were tried: 30 μl, 40 μl and 50 μl per circle of the glass plate. We found that 50 μl final reaction volumes were the most suitable for the well-size (circle) in our experiment. The plates had circles with an internal diameter of 1.9 centimeters. This decision was made based on the amount of liquid that completely filled the circle without spilling outside the circle during mixing with a tooth pick using circular motion. We studied the effect of fetal bovine serum (FBS) concentration in the PBS buffer on the size of the clumps and visibility of the agglutination reaction using 3 different concentrations: 1%, 2% and 3% FBS. We found that 2% FBS was suitable for the SAT. The final reaction contained 20 μl of the sample suspected of containing CPV, 10 μl of buffer (0.2M PBS, pH 7.2, 2% FBS), and 20 μl of 2.5% porcine erythrocytes. We performed all the tests using reagents that were stored in refrigerator and chilled on wet ice in a Styrofoam bucket. The solutions were kept chilled during use. The ambient temperature of virology laboratory is set at 72° C. and is thermostatically controlled. However, the test also performed well on a Brucellosis card test with tear drop shaped wells with chilled reagents. A total of 23 chloroform-extracted fecal suspensions and 60 of cell culture supernatants were tested in SAT. The correct CPV status (positive or negative), and, if positive, the CPV genotype and titer of the virus as determined by conventional plate HA was known for all samples. Further virus isolation was performed on all the fecal samples using the CRFK cell line. After virus isolation, the amount of CPV in the cell supernatant was determined using a plate HA test for CPV. We found a very high correlation (100%, positive/negative status for CPV) between the SAT results with conventional hemagglutination test (Table 1). Only samples with hemagglutination titers on the plate HA test equal to or below <40 were found negative in SAT. It is known that plate HA titer of equal and less than 1:40 is considered negative. Thus, SAT was found to be slightly less sensitive but more accurate in classifying the fecal samples for CPV status. This accuracy is critical because SAT has overcome the low level of false positives that are a serious limitation of the conventional plate HA test. The CPV samples that are less than 1:40 do not react in SAT. When end point titers were compared between SAT and conventional plate HA tests on the same sample, the sensitivity of SAT was lower than that of standard hemagglutination tests. However, the SAT classified the fecal samples correctly on every clinical sample (−23) tested. A total of 60 cell culture samples were tested. Of these, 26 were positive by the conventional plate hemagglutination test and 34 were negative. The SAT missed 6 positive cell culture samples. Thus, for cell culture propagated CPV isolates, we found a lower sensitivity using the SAT assay. This variation is probably due to differences in suitable pH requirement (pH 6.5-7.2) for carnivore parvoviruses propagated in cell culture. Feline panleukopenia viruses require a lower pH of 6.5.

TABLE 1
Correlation between hemagglutination test (HA) titers and slide
agglutination test (SAT) on fecal samples (F, n = 23) and
CRFK cell line (CC, n = 60) supernatants

| Sample No | SAT Result | HA Titre | Sample Type |
|---|---|---|---|
| 1 | Neg | 0 | F |
| 2 | Pos | 320 | F |
| 3 | Neg | 0 | F |
| 4 | Neg | 0 | F |
| 5 | Pos* | 160 | F |
| 6 | Neg | 40 | F |
| 7 | Neg | 0 | F |
| 8 | Neg | 0 | F |
| 9 | Neg | 0 | F |
| 10 | Neg | 40 | F |
| 11 | Neg | 40 | F |
| 12 | Neg | 40 | F |
| 13 | Neg | 0 | F |
| 14 | Neg | 0 | F |
| 15 | Pos* | 80 | F |
| 16 | Neg | 0 | F |
| 17 | Neg | 0 | F |
| 18 | Neg | 0 | F |
| 19^{b} | Neg | 0 | F |
| 20 | Pos | 821920 | F |
| 21 | Neg | 0 | F |
| 22^{a} | Pos | 40960 | F |
| 23 | Neg | 0 | F |
| 24 | Neg | 2560 | CC |
| 25 | Pos | 81920 | CC |
| 26 | Pos | 81920 | CC |
| 27 | Pos | 81920 | CC |
| 28 | Pos** | 40 | CC |
| 29 | Neg | 40960 | CC |
| 30 | Pos | 40960 | CC |
| 31 | Neg | 40960 | CC |
| 32 | Neg | 40 | CC |
| 33 | Neg | 0 | CC |
| 34 | Neg | 0 | CC |
| 35 | Neg | 0 | CC |
| 36 | Pos | 10240 | CC |
| 37 | Neg | 0 | CC |
| 38 | Neg | 0 | CC |
| 39 | Neg | 0 | CC |
| 40 | Pos | 5120 | CC |
| 41 | Pos | 5120 | CC |
| 42 | Neg | 0 | CC |
| 43 | Neg | 0 | CC |
| 44 | Neg | 0 | CC |
| 45 | Pos** | 1280 | CC |
| 46 | Neg | 0 | CC |
| 47 | Neg | 40 | CC |
| 48 | Pos | 5120 | CC |
| 49 | Neg | 0 | CC |
| 50 | Neg | 0 | CC |
| 51 | Neg | 0 | CC |
| 52 | Pos | 20480 | CC |
| 53 | Neg | 320 | CC |
| 54 | Neg | 0 | CC |
| 55 | Neg | 0 | CC |
| 56 | Neg | 0 | CC |
| 57 | Neg | 0 | CC |
| 58 | Pos | 10240 | CC |
| 59 | Neg | 0 | CC |
| 60 | Pos | 10240 | CC |
| 61 | Neg | 0 | CC |
| 62 | Neg | 0 | CC |
| 63 | Neg | 0 | CC |
| 64 | Neg | 5120 | CC |
| 65 | Neg | 0 | CC |
| 66 | Neg | 0 | CC |
| 67 | Neg | 0 | CC |
| 68 | Pos | 5120 | CC |
| 69 | Neg | 0 | CC |
| 70 | Neg | 0 | CC |
| 71 | Neg | 0 | CC |
| 72 | Neg | 0 | CC |
| 73 | Neg | 0 | CC |
| 74 | Pos | 20480 | CC |
| 75 | Pos | 20480 | CC |
| 76 | Neg | 160 | CC |
| 77 | Pos | 20480 | CC |
| 78^{c} | Pos | 20480 | CC |
| 79^{d} | Neg | 0 | CC |
| 80 | Pos | 81920 | CC |
| 81 | Neg | 80 | CC |
| 82 | Pos | 81920 | CC |
| 83 | Neg | 80 | CC |

*Microscopically positive
**Weak positive
^{a}Use as fecal (F) positive control
^{b}Used as fecal (F) negative control
^{c}Used as cell culture (CC) positive control
^{d}Used as cell culture (CC) negative control

In the SIT assay, the presence of antibody was indicated by inhibition of erythrocyte agglutination with hyper immune serum against CPV-2b (Galaxy, Schering Plough Animal Health, Elkhorn Nebr.). We tested CPV-2b (n=1), CPV-2c (n=4) and raccoon parvovirus (n=1) isolates in the SIT assay. The SIT titers of hyperimmune serum were much higher for homologous CPV-2b isolates compared to heterologous CPV-2c isolates. When the hyperimmune anti-CPV-2b serum was used against CPV-2c (07061522), the SIT titer was 1:512. However, when CPV-2b (07080441) was used as the viral antigen, the same hyper immune anti-CPV-2b serum gave an SIT titer of 1:4,096. Thus, the reaction with CPV-2c is about 10-fold lower compared to homologous CPV-2b and anti CPV-2b hyperimmune serum. Moreover, one raccoon parvovirus isolate (08080274) was not inhibited by the standard CPV-2b hyper immune serum indicating that it was antigenically different from CPV isolates.

### Discussion

Canine parvovirus is the number one cause of viral enteritis in dogs and is responsible for significant canine morbidity and mortality (8). Due to the rapid evolution of CPV, monoclonal antibody-based diagnostic tests for field use need to be updated and evaluated for sensitivity against the current variants of CPV in the USA (CPV-2, CPV-2a, CPV-2b, and CPV-2c) (7, 9, 14). Of 148 CPV samples have been genotyped, 13 were CPV-2, 83 were CPV-2b, 68 were CPV-2c, and one was mixed CPV-2b and CPV-2c (9, and S. Kapil unpublished data from OADDL records). There are other reports of mixed CPV-2 infections. Moreover, the cross species transmission of CPV or related viruses further threatens the sensitivity of the monoclonal antibody-based animal side tests that are commercially available for field use. In this study, we have developed generic CPV detection tests for CPV antigen detection in the feces and CPV antibody quantification in serum.

Lateral flow immunoassay (LFA) is a convenient format for front line diagnostics of viruses and antibodies. These tests are specific, sensitive, and easy to use and require minimal training. LFA can be developed for both antigen and antibody detection. However, they require at least one monoclonal or polyclonal antibody to manufacture the test kit. The only limitation of lateral flow assay is that they can be cost prohibitive for routine use when very large numbers of samples have to be tested in kennel situations. Moreover, LFAs are qualitative or semi-quantitative but still very useful screening diagnostic tools.

Enzyme linked immune-sorbent assays for both CPV antigen and antibody have been developed (12, 15), However, ELISAs with even short incubation times (5-10 minutes) require multiple washing steps and can be cost prohibitive for high volume use by kennel breeders for CPV antigen and antibody detection.

Agglutination assays have been tested for CPV and have traditionally used latex beads coated with monoclonal antibodies (20. 22). These assays have been found to be useful for CPV antigen detection. There is also one report of latex beads for antibody detection (1). However, these assays are not commercially available in the Untied States and agglutination tests have been evaluated only in research laboratories (21). Moreover, continuous evolution of CPV affects the usefulness of latex bead assays. There is a need for pan-CPV tests (generic CPV tests) that can detect all genotypes and antigenic variants of CPV for field use. In this study, we have used the intrinsic property of CPV-2 to agglutinate porcine erythrocytes and modified it to develop very rapid, generic, economical assays for CPV antigen and antibody measurements. The only potential limitation of our assay is the need to bleed a pig to obtain erythrocytes. However, it can be solved by properly fixing the swine erythrocytes to provide a longer shelf life at room temperature (11). We have done a preliminary trial with formalin-fixed swine erythrocytes. We found that 4% formalin fixation can adversely affect the performance of SAT for CPV detection (Marulappa and Kapil, unpublished data). Thus, we are trying other fixatives to stabilize the porcine erythrocytes for SAT. In one embodiment, we tried 0.01% sodium azide as a preservative and found that sodium azide does not adversely affect the performance of the test if included in the buffer. Fresh swine erythrocytes can only be used for 1 week and require refrigeration. However, swine erythrocytes can be preserved in sodium azide (0.1%) and thus have a longer shelf life. For hemagglutinating viruses, such as CPV, SAT/SIT can be a very cost effective alternative to field technologies, such as LFA.

We found these twin assays, slide agglutination test/slide inhibition test (SAT/SIT), to be very useful for field applications for the management of CPV outbreaks in kennels. The SIT assay can be used to refine the time of vaccination for CPV puppy shots. For example, if the puppy has high levels of maternal antibodies vaccination can be postponed. However, if there is a low or no antibody titer, the puppy can be vaccinated soon after the negative or low antibody result. CPV antigen and antibody monitoring using these easy and economic assays will lead to better and more timely CPV vaccination compliance and also a more effective “lake” of the CPV vaccine antigens, as vaccination need not occur until after maternal antibodies have dissipated. The SAT assay can also be used to check environmental swipes/swabs for CPV contamination quickly, and if CPV is present, the area can be cleaned and disinfected. The SAT assay can be used to verify the efficiency of decontamination of an area. In addition, even dog and cat erythrocytes that are available in kennels and catteries can be used for SAT/SIT. In some cases, dog and cat erythrocytes are preferred because they further establish the potential host range of e.g. canine parvoviruses. Some earlier (1978) CPV isolates did not infect cats.

We found both of the assays to be practical for kennel use. Because the reagents (PBS and porcine erythrocytes) are commonly available and inexpensive, the assays can be used to monitor CPV in developing countries. The cost per test is very low and all animals can be repeatedly checked for antibodies in the serum against CPV. Whereas the conventional CPV HA test takes 2-4 hours, the present assays for CPV antigen and antibody use the rapid and easy formats of SAT/SIT. Similarly, tests to detect and quantify other significant hemagglutinating viruses, such as influenza A and SARS coronavirus can also be advantageously adapted to SAT/SIT format assays using the buffer and erythrocyte conditions compatible with those viruses. Thus, SAT/SIT can replace the conventional plate hemagglutination tests as quick screening tests. SAT/SIT technology will be useful for any situation where rapid, low cost, and low tech screening is needed for any hemagglutinating virus, for example, during outbreaks in developing countries.

### REFERENCES FOR EXAMPLE 1

1. Boedeus, M., C. Cambiaso, M. Surleraux, and G. Burtonboy. 1988. A latex agglutination-test for the detection of canine parvovirus and corresponding antibodies. J Virol Methods 19:1-12.
2. Buonavoglia, C., V. Martella, A. Pratelli, M. Tempesta, A. Cavalli, D. Buonavoglia, G. Bozzo, G., Elia, N. Decaro, and L. Carmichael. 2001. Evidence for evolution of canine parvovirus type 2 in Italy. J Gen Virol 82:3021-3025.
3. Carmichael, L. E., J. C. Joubert and R. V. H. Pollock. 1980. Hemagglutination by canine parvovirus—serologic studies and diagnostic applications. Am J Vet Res 41:784-791.
4. Cotmore, S. F., and P. Tattersall. 2007. Parvoviral host range and cell entry mechanisms. AdvVirus Res 70: 183-232.
5. Decaro, N., C. Desario, D. D. Addie, Yo. Martella, M. J. Vieira, G. Elia, A. Zicola, C. Davis, G., Thompson, E. Thiry, U. Truyen, and C. Buonavoglia. 2007. Molecular epidemiology of canine parvovirus, Europe. Emerg Infect Diseases 13:1222-1224.
6. Desario, C., N. Decaro, M. Carnpolo, A. Cavalli, F. Cirone, G. Elia, V. Marlena, E. Lorusso, M. Camero, and C. Buonavoglia. 2005. Canine parvovirus infection: Which diagnostic test for virus? J Viral Methods 126:179-185.
7. Hong, C., N. Decaro, C. Desario, P. Tanner, M. C. Pardo, S. Sanchez, C. Buonavoglia, and J. T. Saliki. 2007. Occurrence of canine parvovirus type 2c in the United States. J Vet Diagn Invest 19:535-539.
8. Kapil, S. 1995. Laboratory diagnosis of canine viral enteritis. Current Vet. Therapy 12:697-701.
9. Kapil, S. E. Cooper, C. Lamm, B. Murray, G. Rezabek, L. Johnston, III, G. Campbell, and B. Johnson. 2007. Canine Parvovirus Types 2c and 2b Circulating in North American Dogs in 2006 and 2007. J Clin Microbiol 45:4044-4047.
10. Lopez-Bueno, A., L. P. Villarrea. and J. M. Almendral. 2006. Parvovirus variation for disease: A difference with RNA viruses? CurrTop Microbiol Immunol 299:349-370.
11. Mathys, A., R. Mueller, N. C. Pedersen, and G. H. Theilen. 1983. Hemagglutination with formalin-fixed erythrocytes for detection of canine parvovirus. Am JVet Res 44:150-151.
12. Mildbrand, M. M., Y. A. Teramoto. J. K. Collins, A. Mathys, and S. Winston. 1984. Rapid detection of canine parvovirus in feces using monoclonal antibodies and enzyme-linked immunosorbent-assay. Am Vet Res 45:2281-2284.
13. Mochizuki, M., R. Harasawa and H. Nakatani. 1993. Antigenic and genomic variabilities among recently prevalent parvoviruses of canine and feline origin in Japan. Vet Microbiol 38:1-10.
14. Nakamura, M., K. Nakamura. T. Miyazawa, Y. Tohya, M. Mochizuki, and H. Akashi. 2003. Monoclonal antibodies that distinguish antigenic variants of Canine parvovirus. Clin Diagn Lab Immunol 10:1085-1089.
15. Oh, J. S., G. W. Ha. Y. S. Cho, M. J. Mm, D. J. An, K. K. Hwang, Y. K. lim, B. K. Park B. K. Kang and D. S. Song. 2006. One-step immunochromatography assay kit for detecting antibodies to canine parvovirus. Clin Vac Immunol 13:520-524.
16. Parrish, C. R., and Y. Kawaoka. 2005. The origins of new pandemic viruses: The Acquisition of New Host Ranges by Canine Parvovirus and Influenza A Viruses. Annu Rev Microbiol 59:553-586.
17. Perez, R., L. Francia, V. Romero, L Maya, I. Lopez, and M. Hernandez. 2007. First detection of canine parvovirus type 2c in South America. Vet Microbiol 124:147-152.
18. Pollock, R. V. H., and L E. Carmichael. 1982. Maternally derived immunity to canine parvovirus infection—transfer, decline, and interference with vaccination. J Am Vet Med Assoc 180:37-42.
19. Saknimit, M., I. Inatsuki, Y. Sugiyama, K. Yagami. 1998. Virucidal efficacy of physic-chemical treatments against coronaviruses and parvoviruses of laboratory animals. Jikken Dobutsu. 31: 341-345.
20. Sanekata, T., T. Sugimoto, S. Ueda, M. Tsubokura, Y, Yamane, and M. Senda. 1996. latex agglutination test for canine parvovirus. Aus Vet J 73:215-217.
21. Singh, B. R., R. C, Yadav, S. P. Singh, and V. D. Sharma. 1998. Coagglutination test: A simple and rapid immunodiagnostic test for Parvovirus infection in dogs. Indian J Exp Biol 36:622-624.
22. Veijalainen, P. M. L, E. Neuvonen, A. Niskanen, and T. Juokslahti. 1986. Latex agglutination test for detecting feline panleukopenia virus, canine parvovirus, and parvoviruses of fur animals. J Clin Microbiol 23:556-559.

### Example 2

### SAT Agglutination Tests Conducted at Three Different Temperatures

The dependence of SAT results on temperature of incubation was studied. The CPV samples that were used were all positive samples for CPV in cell culture. SAT tests were conducted as described in Example 1, except that the temperature of incubation was varied. Replica tests were carried out at 4, 21 and 37° C., and the results are presented in Table 2. As can be seen, the SAT reaction can be accurately carried out at room temperature (21° C.), thus making this test even easier to carry out and more amenable to use “in the field”. Also, the CPV-2 isolates that also agglutinate at 37° C. show higher avidity and affinity of reactivity with transferrin receptors. Thus, reactivity at higher temperatures (e.g. 37° C.) is an indirect measure of the pathogenic/virulence potential of the CPV-2 isolates and fitness of the CPV-2 virus. The ability of a CPV-2 isolate to agglutinate RBCs at higher temperatures is a direct and easy measure of its ability to bind to transferrin receptor. Thus, if an isolate (see #5 in this Example) binds at 37° C., binding is also checked as 38, 39, 40, 41, and 42° C. The temperature at which erythrocytes do not lyse but CPV-2 (or other virus) can still bind is an indicator of the binding activity of the virus, in that viruses that bind at higher temperatures bind to transferrin receptor more avidly, and are potentially more virulent that viruses that bind only at lower temperatures. Likewise, the time required for agglutination can also be used to assess the virulence of an isolate, with isolates that cause agglutination quickly generally being more virulent than those that cause agglutination more slowly.

TABLE 2
Agglutination results for CPV at 3 different temperatures

SAT Incubation Temperature

| Tube # | 4° C. | 21° C. | 37° C. |
|---|---|---|---|
| 1 | Neg | Neg | Neg |
| 2 | Pos (M) | Pos (M) | Neg |
| 3 | Pos | Pos | Pos |
| 4 | Pos (M) | Pos (M) | Pos (M) |
| 5 | Pos | Pos | Pos |
| 6 | Neg | Neg | Neg |
| 7 | Neg | Neg | Neg |
| 8 | Neg | Neg | Neg |
| 9 | Pos | Pos | Pos |
| 10 | Pos | Pos | Pos |
| 11 | Pos | Pos | Pos |
| 12 | Pos | Pos | Pos |
| 13 | Pos | Pos | Pos |
| 14 | Pos | Pos | Pos |
| 15 | Pos | Pos | Pos |
| 16 | Pos (W) | Pos (W) | Pos (W) |
| 17 | Pos | Pos | Pos |
| 18 | Pos | Pos | Pos |
| 19 | Neg | Neg | Neg |
| 20 | Pos | Pos | Pos |
| 21 | Pos | Pos | Pos |
| 22 | Pos | Pos | Pos |
| 23 | Pos | Pos | Pos |
| 24 | Pos | Pos | Pos |
| 25 | Pos (M) | Pos (M) | Neg |
| 26 | Pos (M) | Pos (M) | Neg |
| 27 | Pos | Pos | Pos |
| 28 | Pos | Pos | Pos |
| 29 | Pos | Pos | Pos |
| 30 | Pos | Pos | Pos |

Pos is positive, could be seen by naked eye
Pos (M) is positive microscopically
Pos (W) is weak positive
Neg is negative

### Example 3

### Detection of Canine Parvovirus in Biological Fluids Bound to Porcine Erythrocytes by Surface Fluorescence

The assays of the invention were expanded to include detection of CPV in fecal suspensions or fluid samples using surface immunofluorescence (SF). The tests were carried out as follows:

Procedure:

1. Take 50 μl of virus sample (in this case a cell culture sample, case #08060786 chloroform treated, tube #127) in a 1.5 ml centrifuge tube.
2. Add 25 μl of 2.5% porcine erythrocytes and mix by tapping.
3. Incubate for 5 minutes on ice.
4. Add 50 μl of CPV-FITC (CPV virus labeled with fluorescein isothiocyanate) and mix by tapping.
5. Incubate for 20 minutes on ice.
6. Add 500 μl of PBS (pH 7.2 with 2% bovine serum albumin).
7. Wash at 500 rpm for 5 minutes.
8. Discard supernatant.
9. Add 100 μl PBS (pH 7.2, with 2% BSA) and mix.
10. Add 10 μl (in duplicates) on spot slide (having 8 spots) and mount a cover slip.
11. Observe under fluorescent microscope.
Note: Negative control was done with PBS (pH 7.2, with 2% BSA) used in step 1.

It is not possible to detect cell-free virus in biological fluids. The “free” virus must first be bound to a cell or cell surface. Free viruses are too small to see with a fluorescent microscope.

The results are presented in Table 3.

TABLE 3
Results obtained using fluorescent modification
of SAT, or “FSAT”.

| Sample # | FSAT Results | Conventional HA Results |
|---|---|---|
| 1 | Negative | Negative |
| 2 | Negative | Negative |
| 3 | Negative | Negative |
| 4 | Positive | Positive |
| 5 | Negative | Positive* |
| 6 | Positive | Positive |
| 7 | Positive | Positive |
| 8 | Positive | Positive |
| 9 | Negative | Negative |
| 10 | Positive | Positive |
| 11 | Negative | Negative |
| 12 | Negative | Negative |
| 13 | Negative | Negative |

*Weak negative, but became positive upon prolonged incubation for 5 minutes at 4° C.

An analysis of the correlation between the results obtained using FSAT and a conventional plate HA test. The results are presented in Tables 4A-C.

TABLE 4A
Factors for correlation calculation

| | HA Positive | HA Negative | Total |
|---|---|---|---|
| SF Positive | a, True positive | b, False positive | a + b |
| SF Negative | c, False negative | d, True negative | c + d |
| Total | a + c | b + d | a + b + c + d |

TABLE 4B
Data

| Fecal Sample | HA Positive | HA Negative | Total |
|---|---|---|---|
| FSAT Positive | 5 | 0 | 5 |
| FSAT Negative | 1 | 5 | 6 |
| Total | 6 | 5 | 11 |

TABLE 4C
Results

| | |
|---|---|
| Sensitivity = a/(a + c) | 5/6 = 83.33% |
| Specificity = d/(b + d) | 5/5 = 100.00% |
| Accuracy - (a + d)/(a + b + c + d) | 10/11 = 90.91% |

As can be seen, these results show that surface immunofluorescence is about 91% accurate in assessing the presence of CPV. Thus, the tests of the invention can be automated so that results are read and quantitated using surface immunofluorescence.

### Example 4

### Application of SF to High Throughput Screening (FITS) for Antiviral Compounds

SF adapted SAT assays can be used to discover anti-CPV-2 antiviral compounds using HTS techniques. Such tests are used to identity compounds that interfere with the binding of the CPV-2 virus to transferrin, a step that is indicative of the viruses ability to infect cells. Viruses that cannot bind transferrin do not cause agglutination. Testing is carried out using multiwell plates that are UV transparent (e.g. 96 or 346 well plates). Positive control reactions (known CPV-2 sample mixed with erythrocytes and then with fluorescent labeled antibody) are included on each plate. Experimental reactions are carried out by adding a candidate anti-CPV compound to the CPV-2 sample or to the erythrocytes prior to mixing them together. The CPV-2 virus and RBCs are then mixed under conditions that allow the virus to bind to transferrin, unless the compound has interfered with its ability to do so. After washing excess virus from the RBCs, fluorescently labeled antibodies specific for the virus are introduced and will bind to viruses bound to the RBC surface, if any are. Excess labeled antibody is removed (e.g. by washing) and the RBCs are interrogated for fluorescence. A positive signal indicates that a compound is not effective in preventing viral attachment to transferrin. A negative signal indicates that the compound is effective in preventing viral binding to transferrin. A weak signal may indicate that the compound is partially successful in preventing binding. Using the tests of the invention, many compounds (e.g. 500,000 small molecule compound libraries) are assayed at a time using an HTS format.

Candidate compounds that show promise (i.e. negative or weak signal) are tested in cell culture. Successful compounds are then tested in vitro and in vivo for toxicity. The antiviral activity of successful compounds is checked in vivo, typically in 8-week old puppies, followed by challenge with virulent CPV-2 virus. The LD₅₀ and ED₅₀ values are determined in both dogs and cats. Successful compounds can be used to treat or prevent CPV-2 infection, especially in the case of vaccine failure, or unvaccinated puppies.

### Example 5

### Effect of pH of Buffer on Carnivore Parvovirus Positive Samples

Carnivore parvoviruses can be transmitted between dogs and cats, raccoons, foxes, etc. and it is sometimes difficult to know which type of parvovirus is the cause of an outbreak of disease. It had previously been demonstrated that feline and canine parvoviruses preferentially hemagglutinate at different pH values: pH 6.5 for feline and pH 7.2 for canine parvovirus. In order to more precisely identify the source of a parvovirus, the SAT can be performed at pH 6.5 or pH 7.2 or preferably at both pH 6.5 and pH 7.5, and the results can be compared to decide whether a feline or canine virus is implicated.

We performed SAT tests at both pH 6.5 and 7.2 using PBS buffer (0.2M) with fetal bovine serum, as described above. The conditions for the SAT tests were kept the same except the pH was varied. Two virus samples were tested: OADDL 08120386, a parvovirus sample from a cattery that experienced an outbreak of diarrhea, and 08120210, isolated from a dog outbreak. As expected, 08120386 was positive by SAT at pH 6.5 only but not at pH 7.2. However, unexpectedly dog sample 08120210 was positive at pH 7.2 but the clumping improved significantly at pH 6.5 and also was much more rapid at pH 6.5 compared to pH 7.2. Therefore, it is possible that 08120210 was originally a feline CPV and has mutated and gained the ability to partially or slowly agglutinate RBCs at pH 7.2. Alternatively, 08120210 may represent a canine strain of CPV that has mutated, attenuating its ability to hemagglutinate at pH 7.2 but gaining the ability to do so at pH 6.5, thereby expanding its host range. In any case, it is highly likely that this strain, although isolated from a dog, can readily infect cats, and that cats exposed to the CPV strain or at risk for exposure should be vaccinated.

Expanded studies of several recent CPV-2 isolates were carried out with SAT using buffers at pH values of 6.5, 7.2 and 8.0. The results are presented in Table 5. A difference of ≧2 in the degree of hemagglutination was considered to be significant.

TABLE 5
Effect of pH on SAT Results with Current CPV-2 Isolates

| | | Hemagglutination Score | | | |
|---|---|---|---|---|---|
| Sample # | Sample ID | pH 6.5 | pH 7.2 | pH 8.0 | Genotype |
| 21 | 6021051 | ± | negative | negative | CPV-2c |
| 2 | 7030134 | 3+ | 4+ | 5+ | CPV-2b |
| 3 | 7030243 | 1+ | negative | 1+ | CPV-2c |
| 4 | 7030244 | 3+ | 3.5+ | 5+ | CPV-2c |
| 5 | 7030277 | 3+ | 4+ | 5+ | CPV-2b |
| 6 | 7030847 | 3+ | 5+ | 5++ | CPV-2 |
| 7 | 7030850 | 5+ | 5+ | 4+ | CPV-2c |
| 8 | 7040690 | 4+ | 5+ | 5++ | CPV-2b |
| 9 | 7051454 | 3+ | 4+ | 5+ | CPV-2c |
| 29 | 7051346 | 4+ | 3+ | 5+ | CPV-2b |
| 30 | 7051347 | negative | negative | 1+ | CPV-2c |
| 31 | 7061069 | 3.5+ | 3.5+ | 5+ | CPV-2b |
| 33 | 7061069 | 4+ | 2+ | 5+ | ND* |
| 35 | 7061522 | 1+ | 2+ | 3+ | CPV-2c |
| 43 | 7080441 | 1+ | 0.5+ | negative | CPV-2b |
| 54 | 7080797 | 4+ | 4+ | 5+ | CPV-2c |
| 62 | 8051218 | 3+ | 3+ | 4+ | ND |
| 65 | 8051266 | 3.5+ | 6+ | 5+ | ND |
| 66 | 8051267 | 3+ | 3.5+ | 4+ | ND |
| 108 | 8051271 | 3+ | 4+ | 4+ | ND |
| 74 | 8051274 | 4+ | 3+ | 3+ | ND |
| 77 | 8051330 | 4+ | 3+ | 4+ | ND |
| 83 | 8051542 | 4+ | 3+ | 3+ | ND |
| 88 | 8060345 | 1+ | 1+ | 1+ | ND |
| 90 | 8060492 | 3.5+ | 3+ | 3+ | ND |
| 127 | 8060786 | 3+ | 3+ | 4+ | ND |
| 115 | 8060974 | 3+ | 4+ | 3+ | ND |
| 117 | 8060974 | 3+ | 3+ | 3+ | ND |
| 129 | 8070153 | negative | negative | 1+ | ND |
| 131 | 8071346 | 5+ | 4+ | 3+ | ND |
| 130 | 8071352 | 5+ | 5+ | 4+ | ND |
| 150 | 8080274 | 3+ | 3+ | 3+ | ND |
| | 8080274 | 4+ | 3.5+ | 3.5+ | ND |
| 135 | 8090386 | 4+ | 4+ | 4+ | ND |

*ND = not done

As can be seen, recent CPV-2 isolates have evolved to differ in the degree of hemagglutination at pH values of 6.5, 7.2 and 8.0, and, contrary to earlier findings, there is a tendency in newer CPV-2 viruses for pH 8.0 to be more suitable for hemagglutination than pH 7.2.

These differences are likely due to differences in the net charge among isolates, which likely affects virus-receptor interaction. Of note, an increase in pH optimum would likely permit improved virus-receptor interaction in, for example, the small and large intestine where the pH tends to be alkaline, and would thus likely translate into increased virulence of the virus. Thus, advantages may accrue by checking virus samples using SAT at several pH values, e.g. in order to ascertain the virulence of a virus in vitro.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for detecting the presence of a red blood cell (RBC) agglutinating virus in a sample, comprising the steps of
mixing, on the surface of a substrate, a microliter quantity of said sample with a microliter quantity of a RBC solution to form a reaction mixture with a final volume of 1000 μl or less; and
incubating said reaction mixture for one minute or less; and
detecting in said reaction mixture on said surface of said substrate the presence or absence of RBC agglutination, wherein the presence of RBC agglutination indicates the presence of a RBC agglutinating virus in said sample, and the absence of RBC agglutination indicates the absence of a RBC agglutinating virus in said sample.

2. The method of claim 1, wherein said sample is a fecal sample.

3. The method of claim 1, wherein said RBC agglutinating virus is a canine parvovirus.

4. The method of claim 1, wherein said final volume is 50 μl.

5. The method of claim 1, wherein said step of detecting is carried out by visually inspecting said reaction mixture on said surface of said substrate.

6. The method of claim 1, wherein said RBCs in said RBC solution are of porcine origin.

7. The method of claim 1, wherein said step of detecting is carried out by surface immunofluorescence.

8. The method of claim 7, wherein said method is carried out using a high throughput screening (HTS) format.

9. The method of claim 1, wherein said method is carried out at one or more pH values selected from the group consisting of pH 6.5, pH 7.2 and pH 8.0.

10. A method for detecting the presence of a antibodies to a red blood cell (RBC) agglutinating virus in a sample, comprising the steps of
exposing said RBC agglutinating virus to said sample;
mixing, on the surface of a substrate, a microliter quantity of a solution containing said exposed virus with a microliter quantity of a RBC solution to form a reaction mixture with a final volume of 1000 μl or less;
incubating said reaction mixture for one minute or less; and
detecting in said reaction mixture on said surface of said substrate the presence or absence of RBC agglutination, wherein the presence of RBC agglutination indicates the absence of antibodies to said RBC agglutinating virus in said sample, and the absence of RBC agglutination indicates the presence of antibodies to said RBC agglutinating virus in said sample.

11. The method of claim 10, wherein said sample is a blood or serum sample.

12. The method of claim 10, wherein said RBC agglutinating virus is a canine parvovirus.

13. The method of claim 10, wherein said final volume is 50 μl.

14. The method of claim 10, wherein said step of detecting is carried out by visually inspecting said reaction mixture on said surface of said substrate.

15. The method of claim 10, wherein said RBCs in said RBC solution are of porcine origin.
